(19) United States
(12) Patent Application Publication
(10) Pub. No.: US 2004/0244796 A1
(43) Pub. Date: Dec. 9, 2004

(54) Regulator for underwater breathing apparatus

(75) Inventor: Roberto Semeia, Lavagna (IT)

Correspondence Address: FRANCO A. SERAFINI, 2455 CAMINITO CARTGATE, LA JOLLA, CA, 92037, US

(73) Assignee: SCUBAPRO EUROPE SRL, Casarza Ligure (IT)

(21) Appl. No.: 10/859,397

(22) Filed: Jun. 2, 2004

(30) Foreign Application Priority Data
Jun. 6, 2003 (IT) IT-SV2003A000026

(51) Int. Cl. A61M 16/00

(52) U.S. Cl. 128/204.26; 128/204.18

(57) ABSTRACT

A regulator for underwater breathing apparatus comprising a first stage regulator, a second stage regulator, and a hose, wherein one or more thermally conductive inserts are positioned between the first stage regulator, the second stage regulator, and the hose, causing a heat transfer between the surrounding water and the breathing gas. This invention further relates to a regulator for underwater breathing apparatus wherein breathing resistance is decreased or eliminated by providing one or more expansion tanks that store breathing gas and that are positioned between the first stage regulator, the second stage regulator, and the hose. In one embodiment, the thermally conductive inserts also act as expansion tanks.

### CROSS-REFERENCE TO RELATED APPLICATIONS

This application claims the benefit of foreign application SV2003A000026, filed in Italy on Jun. 6, 2003 and titled “Erogatori per autorespiratori per uso subacqueo.”

### STATEMENT REGARDING FEDERALLY SPONSORED RESEARCH OR DEVELOPMENT

Not Applicable.

### REFERENCE TO A SEQUENCE LISTING, A TABLE, OR A COMPUTER PROGRAM LISTING COMPACT DISK APPENDIX

Not Applicable.

### BACKGROUND OF THE INVENTION

1. Field of the Invention

The present invention relates to a regulator for underwater breathing equipment. In different embodiments, the regulator increases the temperature of the breathing gas by means of thermally conductive inserts transferring heat from the water to the gas, or facilitates the breathing action of the diver by storing gas in expansion tanks, or both.

2. Description of Related Art

Pressure regulators for underwater diving typically comprise a first stage regulator, that reduces breathing gas pressure from the higher cylinder pressure to an intermediate level; a second stage regulator, that reduces breathing gas pressure from the intermediate level to the lower breathing pressure of approximately one bar; and a hose, that sealingly connects the outlet of the first stage to the inlet of the second stage.

When diving in conditions of extreme cold, the temperature of the surrounding water is above freezing point. Instead, the breathing gas may drop to temperatures below freezing point, due to the expansion of the gas within the cylinder as gas is being withdrawn, and to the expansion consequent to a reduction in gas pressure from cylinder pressure to breathing pressure.

Such a reduction in temperature may cause condensation of the moisture present in the breathing gas and may cause water or even ice deposits in certain parts of the first and of the second stage pressure regulators or in the ducts of the pressure regulator, with a negative effect on the proper operation of the regulator. In extreme cases, ice causes the regulator to freeze up; in other cases, the diver may undergo a respiratory shock. In some cases, the effect of the reduction in gas temperature may be so severe to cause the death of the diver.

Therefore, there is a need to compensate the temperature of the breathing gas as it expands when pressure is reduced. Some regulators have been introduced in the prior art to resists the effect of reduction of gas temperatures. For instance, some regulators have certain components that are specially coated to prevent ice from sticking. Other regulators are environmentally sealed to avoid penetration and subsequent freezing of external water.

An additional drawback of regulators in the prior art is the suction effort required of the diver to receive an adequate volume of air according to volumetric lung demand. In some instances, breathing has been facilitated by using large diameter hoses, which reduce breathing resistance. Unfortunately, large diameter hoses are not readily available on the market as standard parts, are inconvenient because bulky, and are stiffer than small diameter hoses. The increased stiffness on large hoses is discharged on the second stage regulator, to which the mouthpiece is connected, causing the mouthpiece to move and, therefore, making it more difficult for the diver to retain the mouthpiece in position.

Therefore, there is an additional need for an improved regulator where gas is heated when it expands due to pressure reductions, and where inhalation is smoother and more similar to normal physiological conditions, in order to reduce or eliminate an active suction effort. Further, there is a need to eliminate the drawbacks caused by large diameter hoses, and, in general, to allow the use of smaller diameter hoses, which are more flexible.

### BRIEF SUMMARY OF THE INVENTION

This invention relates to a regulator for underwater breathing apparatus, and more specifically to a regulator wherein one or more thermally conductive inserts are positioned between the outlet of the first stage regulator and the inlet of the second stage regulator, causing a heat transfer between the surrounding water and the breathing gas. Such thermally conductive inserts may be separate components, for instance, fittings interposed between the outlet of the first stage regulator, the inlet of the second stage regulator, and the hose, or may instead be designed as integral parts of the first stage regulator, the second stage regulator, or the hose.

This invention further relates to a regulator for underwater breathing apparatus wherein breathing resistance is decreased or eliminated by providing one or more expansion tanks that store breathing gas. Such expansion tanks may be shaped externally like fittings that can be interposed between the first stage regulator, the second stage regulator, and the hose, and internally like tubular chambers. Further, such expansion tanks may be manufactured as a single piece or from a plurality of pieces.

In one embodiment, the thermally conductive inserts also act as expansion tanks.

It is one advantage of the present invention to provide heat compensation to the breathing gas between the first stage regulator and the second stage regulator.

It is another advantage of the present invention to reduce breathing resistance of the diver by providing a greater volume of gas in the section between the first stage regulator and the second stage regulator.

It is a further advantage of the present invention to provide expansion tanks between the first stage regulator and the second stage regulator that can be shaped in a variety of ways, for instance, like fittings, straight tubular chambers, or tubular chambers with different angles, for instance, elbow-shaped.

It is yet another advantage of the present invention to allow for the use of regulator hoses of smaller diameter than in the prior art.

Other objects and advantages of the present invention will become apparent from the following descriptions, taken in connection with the accompanying drawings, wherein, by way of illustration and example, embodiments of the present invention are disclosed.

### BRIEF DESCRIPTION OF THE SEVERAL VIEWS OF THE DRAWINGS

The drawings constitute a part of this specification and include exemplary embodiments of the invention, which may be embodied in various forms. It is to be understood that in some instances various aspects of the invention may be shown exaggerated or enlarged to facilitate an understanding of the invention.

FIG. 1 is a perspective view of one embodiment of the invention, comprising a first stage regulator, a second stage regulator, a hose and three expansion tanks.

FIG. 2 is a perspective sectional view of an expansion tank manufactured as a one-piece fitting.

FIG. 3 is a perspective sectional view of an expansion tank manufactured as a two-piece fitting, wherein the two pieces are sealingly joined together.

### DETAILED DESCRIPTION OF THE INVENTION

Detailed descriptions of embodiments of the invention are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

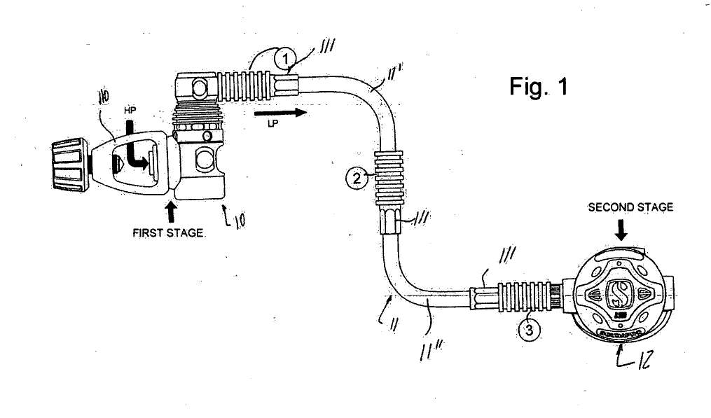

With reference to FIG. 1, a regulator for underwater breathing apparatus typically comprises a first stage regulator 10, which reduces gas pressure from a higher source pressure to an intermediate pressure (generally, 9-10 bars); a second stage regulator 12, which reduces gas pressure from the intermediate pressure to a lower pressure compatible with human breathing (approximately 1 bar); and a hose 11, which transfers gas from first stage regulator 10 to second stage regulator 12.

First stage regulator 10 comprises an inlet, which is sealingly connected by an attachment 110 to a pressurized source of gas, typically a cylinder containing air or another breathing mixture suitable for scuba diving. Further, first stage regulator 10 comprises an outlet, which is connected to one end of hose 11 and through which gas at the intermediate pressure exists first stage regulator 10. Conversely, second stage regulator 12 comprises an inlet, which is connected to the other end of hose 11, and an outlet, which is connected to the diver's mouthpiece.

In a first embodiment of the invention, three expansion tanks 1, 2, and 3 are connected to hose 11 to store gas. In other embodiments, only one or two expansion tanks may be provided.

Expansion tanks 1, 2, and 3 may be of any volume. Preferably, expansion tanks 1, 2, and 3 have a total capacity substantially corresponding to the volumetric lung demand, that is, to the volumetric inhalation demand of the diver's lungs at breathing pressure. Nevertheless, expansion tanks 1, 2, and 3 may be designed to store different amounts of gas in order to achieve certain size and weight ratios. For instance, each one of expansion tanks 1, 2, and 3 may store an amount of gas substantially corresponding to the volumetric lung demand at breathing pressure, or the total capacity of expansion tanks 1, 2, and 3 may be less than the volumetric lung demand at breathing pressure, or the total capacity of expansion tanks 1, 2, and 3 may be substantially equal to the volumetric lung demand but each one of expansion tanks 1,2, and 3 may be of different sizes.

Embodiments of the invention comprising only one or two expansion tanks will include expansion tanks of different sizes than in embodiments with three expansion tanks that provide comparable performance. As an example of the capacity of the expansion tanks, in an embodiment with a single expansion tank, if the pressure inside the expansion tank is about 9.5 bars, and if the volumetric lung demand at breathing pressure is considered to be about 1.5 liters at the inhalation pressure of 1 bar, the required capacity of the expansion tank is 0.15 liters. That means that the expansion tank has a relatively small volume, and expansion tanks of even smaller volumes will be required if more than one expansion tank is employed.

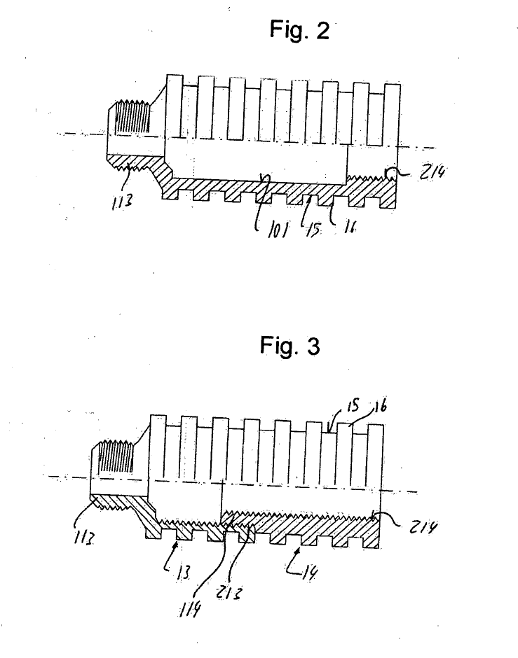

Expansion tanks 1, 2, and 3 may be positioned at different points between first stage regulator 10 and second stage regulator 12 and have different shapes. As shown in FIGS. 2 and 3, expansion tanks 1, 2, and 3 may be internally shaped as tubular chambers and externally shaped as fittings. Further, expansion tank 1 may be connected directly to first stage regulator 10 and expansion tank 2 to second stage regulator 12. Expansion tank 3 may instead be positioned along hose 11 by designing hose 11 in two segments 11′ and 11″ connected by expansion tank 3. In order to operate as fittings, expansion tanks 1, 2, and 3 are designed to comprise an outward-projected threaded end (“male end”) 113 and an inward-projected threaded end (“female end”) 214, which allows for the use of conventional first stage regulators, second stage regulators, and hoses.

When one of expansion tanks 1, 2, and 3 is connected directly to the first stage regulator or to the second stage regulator, the added advantage is achieved of providing a rigid support to that expansion tank.

In general, while it is desirable to position an expansion tank directly at the second stage inlet, in order to make the breathing action of the diver smoother and psychologically similar to normal breathing conditions, size and weight considerations would make it preferable to position an expansion tank 1 directly at the first stage outlet, because size and weight are less critical in the proximity of first stage regulator 10, which is connected to the cylinder, than in the proximity of second stage regulator 12, which is retained by the diver's mouth.

Therefore, a configuration as shown in FIG. 1 is advantageous, because expansion tank 1 is directly connected to first stage 10 and expansion tank 2 is directly connected to second stage 12, or, in a configuration with only two expansion tanks, one tanks is preferably connected directly to first stage 10 and the other directly connected to second stage 12. In either case, the expansion tanks may be designed with a total capacity corresponding to the volumetric lung demand, and such total capacity may be apportioned among the tanks in equal or different percentages, for instance, the expansion tank next to first stage regulator 10 may be of a larger capacity than the expansion tank next to second stage 12.

The expansion tanks may have straight or angled shapes. For instance, when expansion tank 3 is angled to an elbow shape, hose 11 is connected to second stage 12 in a direction parallel to the diver's mouth and not perpendicularly. This is advantageous because the spring-back forces exerted by hose 11 are then not exerted perpendicularly to the mouthpiece, that is, do not pull the mouthpiece away from the diver's mouth, but are instead transversal to the axis of the mouthpiece, that is, they are applied at a slight offset of the mouthpiece in relation to the mouth and allow for better control of the mouthpiece, requiring a lower muscular effort of the diver.

Turning now to FIGS. 2 and 3, the expansion tanks that are part of the present invention may be provided in a single piece or in a plurality of pieces that are sealably and removably joined together. Both expansion tanks shaped like straight fittings and expansion tanks shaped like angled fittings may be single- or multi-piece. Further, expansion tanks shaped externally like fittings may be shaped internally like tubular chambers, of constant or of varying diameters.

Specifically, FIG. 2 illustrates an expansion tank manufactured as a one-piece fitting. Instead, FIG. 3 illustrates an expansion tank comprising two tubular elements, a first tubular element 13 and a second tubular element 14. As shown in FIG. 3, first tubular element 13 is cup-shaped and includes male end 113, which can be screwed, for instance, to the second stage inlet, and a first threaded portion 213, which typically has a larger diameter than male end 113. Second tubular element 14 instead comprises a second threaded portion 114, which exhibits a threaded pattern on a part of its external surface that enables a mating connection with first threaded portion 213, and female end 214, which enable a connection, for instance, with one end of hose 11.

In another embodiment, some or all of the expansion tanks may also serve as thermally conductive inserts, that is, the expansion tanks may be designed to increase gas temperature by transferring heat from the external water to the gas. That is desirable because the external water is generally warmer than the gas fed to the diver, and because the expansion of the gas, both in the cylinder and after the reduction in pressure occurring after the first stage regulator, may cause gas temperature to drop below zero degrees centigrade, especially when operating al the low end of the diving temperature range.

While expansion tanks may be manufactured from any suitable material, in order to enhance heat transfer it is desirable to manufacture some or all of the expansion tanks with a material that is both light and mechanically strong, and that is thermal conductive. Examples of such materials are metals, such as aluminium alloys; plastics with higher than average conductivity; and plastics containing fillers, for instance, plastics with conductive fillers such as graphite powder.

When more than one expansion tank is present, the expansion tanks may be manufactured of different materials of different strengths and of different thermal conductivities. For instance, when three expansion tanks are present, expansion tank 1 (connected to the first stage regulator) may be made of metal, while expansion tanks 2 and 3 may be made of plastic. In this configuration, expansion tank 1 is made of a heavier material and is supported by the first stage regulator, which is directly connected to the cylinder, making weight less critical than is other parts of the regulator, whereas a lighter material is used for the other tanks. The lower thermal conductivities of expansion tanks 2 and 3 are compensated by the higher thermal conductivity of expansion tank 1, while at the same time the lighter weight of expansion tank 2 decreases the effort required by the diver to retain the mouthpiece into position.

The expansion tanks shown in FIGS. 2 and 3 may be manufactured employing a molding or a machining process. For instance, the expansion tank of FIG. 3 may be manufactured by milling first tubular element 13 and second tubular element 14, and because second threaded portion 114 is screwed into first threaded portion 213, by milling the inner diameter of second tubular element 14 with a drill bit of a smaller diameter than the inner diameter of first threaded portion 213.

Further, in order to increase the heat transfer rate between the outer water and the inner gas, the expansion tanks may be equipped with fins providing a greater heat transmission surface. Such fins may have a variety of shapes, for instance, be wave-shaped, rib-shaped, or be shaped like corrugations. Additionally, these fins may cover all or only portions of the external surfaces of the expansion tanks and may be an integral part of the expansion tanks, or may be separate elements affixed to the expansion tanks.

The fins may be an integral part of the expansion tanks, or be separate components affixed to the expansion tanks. The fins may also be manufactured through a milling or a molding process.

As a way of illustration, FIGS. 1, 2, and 3 show expansion tanks 1, 2, and 3 with fins shaped like annular crests 16 that extend from the outer surfaces of the expansion tanks and that are separated by annular grooves 15. In this example, annular crests 16 and annular grooves 15 have the same longitudinal thickness, but different thicknesses may be employed in different embodiments.

Because expansion tanks increase the amount of gas stored inside a regulator, hoses with smaller diameters than in the prior art may be employed. This enables to diver to use hoses or hose segments that are less bulky, more easily deformable and that exert lower spring-back forces than in the prior art. In general, the hose will have a reduced diameter compared to the expansion tanks. The ratio of the hose diameter to the diameter of the expansion tanks will be determined according to design and performance considerations, such as the desired volume of stored gas and the desired gas flow.

Other embodiments of the present invention are possible, wherein the expansion tanks may be designed to perform only a gas storage function and not a heat transferring function, for instance, by employing materials and shapes that are less conducive to heat transfer. Conversely, heat transferring inserts may be employed that are not designed to operate as gas storage elements, for instance, by selecting inner diameters that are not substantially different from the hose diameter. Both the expansion tanks and the heat transferring inserts may be provided shaped like fittings and like tubular sleeves, in the manner described above.

However, the integration of the gas storage and heat transfer functions provides a construction that is simpler, cheaper and less bulky than employing different elements with separate functions.

In other embodiments, the expansion tanks and/or the thermally conductive inserts may be designed to be integral elements of the first stage outlet, of the second stage inlet, or of one or both ends of hose 11 or of hose segments 11′ and 11″.

While the invention has been described in connection with a number of embodiments, it is not intended to limit the scope of the invention to the particular forms set forth, but on the contrary, it is intended to cover such alternatives, modifications, and equivalents as may be included within the spirit and scope of the invention as defined by the appended claims.

What is claimed is:

1. A regulator for underwater breathing apparatus comprising:
a first stage regulator for reducing gas pressure from a higher level to an intermediate level, the first stage regulator having a first stage inlet and a first stage outlet;
a second stage regulator for reducing gas pressure from the intermediate level to a lower level compatible with human breathing, the second stage regulator having a second stage inlet and a second stage outlet;
a hose for transferring gas from, the first stage outlet to the second stage inlet; and
one or more thermally conductive inserts, positioned between the first stage outlet and the second stage inlet,
wherein the one or more thermally conductive inserts operate as heat conductors between the gas and the surrounding water.

2. The regulator for underwater breathing apparatus of claim 1, wherein at least one of the one or more thermally conductive inserts is an integral part of the first stage regulator, the second stage regulator, or the hose.

3. The regulator for underwater breathing apparatus of claim 1, wherein at least one of the one or more thermally conductive inserts is provided as a fitting.

4. The regulator for underwater breathing apparatus of claim 3, wherein the fitting may be sealably connected to the first stage outlet, the second stage inlet or the hose.

5. The regulator for underwater breathing apparatus of claim 3, wherein the hose is divided in segments, each couple of segments being connected by the fitting.

6. The regulator for underwater breathing apparatus of claim 3, wherein the one or more thermally conductive inserts are shaped as tubular sleeves.

7. The regulator for underwater breathing apparatus of claim 6, wherein the tubular sleeves have a straight shape.

8. The regulator from underwater breathing apparatus of claim 6, wherein the tubular sleeves have angled shapes.

9. The regulator from underwater breathing apparatus of claim 1, wherein at least one of the one or more thermally conductive inserts is made of metal.

10. The regulator from underwater breathing apparatus of claim 1, wherein at least one of the one or more thermally conductive inserts is made of plastic.

11. The regulator from underwater breathing apparatus of claim 1, wherein at least one of the one or more thermally conductive inserts is made of plastic with a thermally conductive filler.

12. The regulator from underwater breathing apparatus of claim 1, wherein the one or more thermally conductive inserts comprise heat transferring fins on their outer surface.

13. The regulator from underwater breathing apparatus of claim 12, wherein the heat transferring fins are integral parts of the one or more thermally conductive inserts.

14. The regulator from underwater breathing apparatus of claim 12, wherein the heat transferring fins are separate elements affixed to the outer surface of the one or more thermally conductive inserts.

15. The regulator from underwater breathing apparatus of claim 12, wherein the heat transferring fins are wave-shaped.

16. The regulator from underwater breathing apparatus of claim 12, wherein the heat transferring fins are rib-shaped.

17. The regulator from underwater breathing apparatus of claim 12, wherein the heat-transferring fins are corrugations on the outer surface of the one or more thermally conductive inserts.

18. A regulator for underwater breathing apparatus comprising:
a first stage regulator for reducing gas pressure from a higher level to an intermediate level, the first stage regulator having a first stage inlet and a first stage outlet;
a second stage regulator for reducing gas pressure from the intermediate level to a lower level compatible with human breathing, the second stage regulator having a second stage inlet and a second stage outlet;
a hose for transferring gas from the first stage outlet to the second stage inlet; and
one or more expansion tanks, positioned between the first stage outlet and the second stage inlet,
wherein the one or more expansion tanks store gas.

19. The regulator for underwater breathing apparatus of claim 18, wherein the one or more expansion tanks store an amount of gas corresponding to at least a fraction of the volumetric lung demand at breathing pressure during a single inhalation.

20. The regulator for underwater breathing apparatus of claim 18, wherein the one or more expansion tanks store an amount of gas substantially corresponding to the volumetric lung demand at breathing pressure during a single inhalation.

21. The regulator for underwater breathing apparatus of claim 18, wherein a single expansion tank is provided.

22. The regulator for underwater breathing apparatus of claim 18, wherein one of the one or more expansion tanks is directly connected to by the first stage regulator, the second stage regulator, or the hose.

23. The regulator for underwater breathing apparatus of claim 18, wherein the hose is divided in segments, each couple of segments being connected by one of the one or more expansion tanks.

24. The regulator for underwater breathing apparatus of claim 18,
wherein the one or more expansion tanks are a plurality of expansion tanks,
wherein one of the expansion tanks is directly connected to the first stage regulator, and
wherein one of the expansion tanks is directly connected to the second stage regulator.

25. The regulator for underwater breathing apparatus of claim 24, wherein the hose is divided in segments, each segment being connected to the next segment by one of the expansion tanks.

26. The regulator for underwater breathing apparatus of claim 18, wherein the one or more expansion tanks are internally shaped as tubular chambers and externally shaped as fittings.

27. The regulator for underwater breathing apparatus of claim 26, wherein at least one of the one or more expansion tanks has a straight shape.

28. The regulator for underwater breathing apparatus of claim 26, wherein at least one of the one or more expansion tanks has an angled shape.

29. The regulator for underwater breathing apparatus of claim 28, wherein one of the expansion tanks having an angled shape is directly connected to the second stage regulator.

30. The regulator for underwater breathing apparatus of claim 18, wherein at least one of the one or more expansion tanks comprises two or more tubular elements.

31. The regulator for underwater breathing apparatus of claim 30, wherein the tubular elements are cup-shaped and sealingly joined to each other.

32. The regulator for underwater breathing apparatus of claim 18, wherein the one or more expansion tanks are manufactured by a milling process.

33. The regulator for underwater breathing apparatus of claim 18, wherein the one or more expansion tanks are manufactured by a molding process.

34. The regulator for underwater breathing apparatus of claim 18, wherein the one or more expansion tanks are made of metal.

35. The regulator for underwater breathing apparatus of claim 18, wherein the one or more expansion tanks are made of plastic.

36. The regulator for underwater breathing apparatus of claim 18, wherein the one or more expansion tanks are made of plastic with a filler.

37. The regulator for underwater breathing apparatus of claim 18, further comprising one or more thermally conductive inserts that are integrated with the one or more expansion tanks.

38. The regulator for underwater breathing apparatus of claim 37, wherein the one or more expansion tanks have inner cavities with sections of different diameters.

39. The regulator for underwater breathing apparatus of claim 37, wherein the one or more expansion tanks form single components with the one or more thermally conductive inserts.

40. The regulator for underwater breathing apparatus of claim 37, wherein the one or more expansion tanks have capacities.

41. The regulator for underwater breathing apparatus of claim 37,
wherein the one or more expansion tanks are a plurality of expansion tanks,
wherein one of the plurality of expansion tanks is directly connected to the first stage and is made of metal, and
wherein another one of the plurality of expansion tanks is directly connected to the second stage and is made of plastic.

42. The regulator for underwater breathing apparatus of claim 37, wherein the one or more thermally conductive inserts are a plurality of inserts with different thermal conductivities.

43. The regulator for underwater breathing apparatus of claim 37, wherein the one or more thermally conductive inserts comprise heat transferring fins on at least part of their outer surface.

44. The regulator for underwater breathing apparatus of claim 43, wherein the heat transferring fins are integral parts of the one or more thermally conductive inserts.

45. The regulator for underwater breathing apparatus of claim 43, wherein the heat transferring fins are separate elements affixed to the outer surface of the one or more thermally conductive inserts.

46. The regulator for underwater breathing apparatus of claim 43, wherein the heat transferring fins are wave-shaped.

47. The regulator for underwater breathing apparatus of claim 43, wherein the heat transferring fins are rib-shaped.

48. The regulator for underwater breathing apparatus of claim 43, wherein the heat-transferring fins are formed by corrugations on the outer surface of the one or more thermally conductive inserts.

49. The regulator for underwater breathing apparatus of claim 37, wherein the hose has a reduced diameter compared to the one or more expansion tanks, the ratio of the hose diameter to the diameters of the one or more expansion tanks being determined by the desired volume of stored gas and of gas flow.
